(19) United States
(12) Patent Application Publication
(10) Pub. No.: US 2022/0149656 A1
(43) Pub. Date: May 12, 2022

(54) DEVICE FOR CHARGING ELECTRONIC DEVICES AND SANITIZING SURFACES

(71) Applicant: Molonlave Group LLC, Long Island City, NY (US)

(72) Inventor: Marios Georgiades, Saddle River, NJ (US)

(21) Appl. No.: 17/525,359

(22) Filed: Nov. 12, 2021

Related U.S. Application Data
(60) Provisional application No. 63/112,998, filed on Nov. 12, 2020.

(51) Int. Cl. H02J 50/00; A61L 2/10

(52) U.S. Cl. CPC H02J 50/005; A61L 2/10

(57) ABSTRACT

An electronic device includes a housing, a battery in the housing, a power transmitter in the housing configured for wireless charging of a second electronic device; and at least one light source emitting ultraviolet-C (UV-C) light. The UV-C light comprises a wavelength in a range of 270 nm to about 280 nm.

### FIELD

The field relates generally to a device used to sanitize surfaces and to charge electronic devices, such as, for example, portable electronic devices, including but not necessarily limited to, mobile phones, smartphones, and tablets.

### BACKGROUND

Portable personal electronic devices have become ubiquitous, and are used by many individuals on a daily basis. Such personal electronic devices may include mobile phones, smartphones, and tablet computers. It is estimated that 5 billion people worldwide have mobile devices. Individuals use such devices to access a variety of mobile applications such as, social media applications, online video and audio streaming applications, and mobile gaming applications. Portable personal electronic devices typically come equipped with rechargeable battery technology such that a user need not replace their batteries when they become discharged. Such battery technology can be recharged using battery chargers, such as, for example, wall outlet chargers and portable charging devices. However, such portable charging devices are typically cumbersome and require a cable connecting the portable personal electronic device and the portable charging device.

Also, given the recent pandemic attributed to Coronavirus (COVID-19), individuals have become increasingly concerned about maintaining their health and staying free of germs that may transmit viruses. Often times, after touching surfaces, people wash their hands or use alcohol-based sanitizer to kill virus-carrying germs or other contaminants with which they may have come in contact. Although hand-washing and sanitizer may be effective at killing viruses, people may not always have sinks and/or sanitizer at their disposal, or they may seek alternative and/or additional means of protection.

### SUMMARY

Illustrative embodiments of the invention provide a portability device configured to perform portable recharging of electronic devices and sanitizing of surfaces.

According to an embodiment, an electronic device includes a housing, a battery in the housing, a power transmitter in the housing configured for wireless charging of a second electronic device; and at least one light source emitting ultraviolet-C (UV-C) light.

According to another embodiment, an electronic device includes a connector for connecting to a second electronic device, a battery configured for charging another battery of the connected second electronic device, and at least one light source emitting UV-C light.

The UV-C light comprises a wavelength in a range of 270 nm to about 280 nm.

### BRIEF DESCRIPTION OF THE DRAWINGS

FIG. 1 shows a front view of a charging and sanitizing device in accordance with an embodiment.

FIG. 2 shows a bottom perspective view of the charging and sanitizing device in accordance with an embodiment.

FIG. 3 shows a left perspective view of the charging and sanitizing device in accordance with an embodiment.

FIG. 4 shows a left side view of the charging and sanitizing device in accordance with an embodiment.

FIG. 5A shows light sources mounted on a substrate in accordance with an embodiment.

FIG. 5B shows a face portion of the housing including openings in which the light sources are disposed in accordance with an embodiment.

FIG. 6 shows a perspective view of the charging and sanitizing device attached to a portable personal electronic device in accordance with an embodiment.

FIG. 7 shows some internal components of the charging and sanitizing device in accordance with an embodiment.

FIG. 8 shows a front view of a charging and sanitizing device in accordance with another embodiment.

FIG. 9 shows a left perspective view of the charging and sanitizing device in accordance with another embodiment.

FIG. 10 shows a top perspective view of the charging and sanitizing device in accordance with another embodiment.

FIG. 11 depicts a sanitizing operation of a charging and sanitizing device in accordance with an embodiment.

FIG. 12 shows a front view of a charging device in accordance with an embodiment.

FIG. 13 shows a right side view of the charging device in accordance with an embodiment.

FIG. 14 shows a right perspective view of the charging device in accordance with an embodiment.

FIG. 15 shows a bottom perspective view of the charging device in accordance with an embodiment.

### DETAILED DESCRIPTION

Illustrative embodiments provide a non-cumbersome, portable charging device that can recharge portable personal electronic devices, while maintaining a hands-free user experience. In addition, one or more embodiments provide an electronic surface sanitizing device, which is also a charging device. As detailed herein, one or more embodiments provide a device that can be attached to the rear surface of a personal electronic device to provide wireless recharging of the personal electronic device. In some embodiments, a charging device includes ultra violet-C (UV-C) light sources, which can be used to disinfect surfaces.

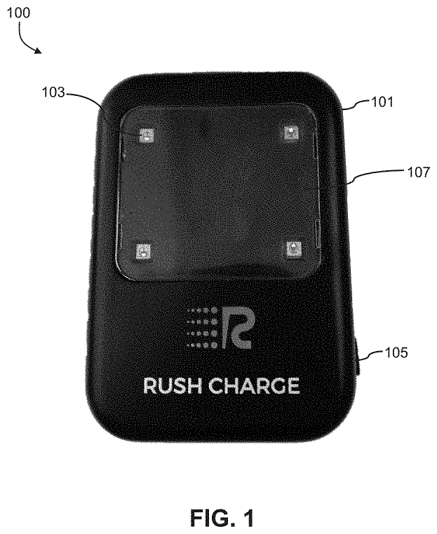

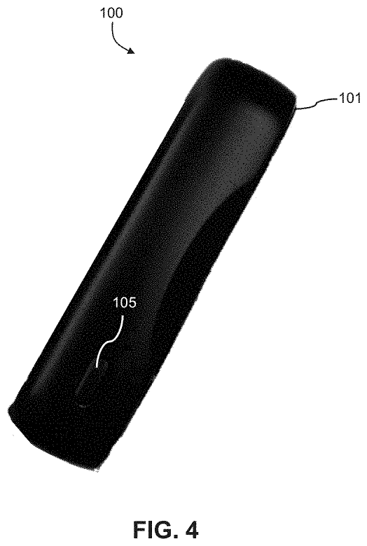

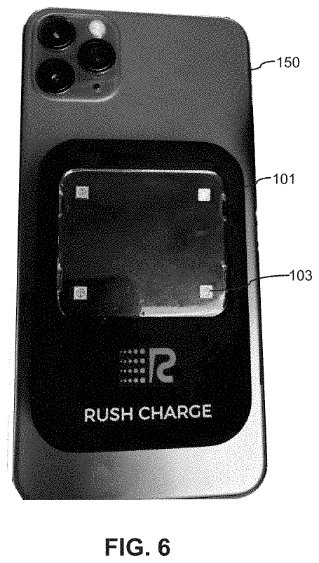

FIGS. 1-7 illustrate a device 100, which comprises a housing 101 and a plurality of light sources 103 emitting UV-A and/or UV-C light. In an illustrative embodiment, the device 100 is configured to perform wireless charging of electronic devices, including but not necessarily limited to, mobile phones, smartphones or tablets in accordance with standards for wireless power transfer (“Qi”) set forth by the Wireless Power Consortium (WPC). See, e.g., WPC, The Qi Wireless Power Transfer System—Power Class 0 Specification, Version 1.2.3 (February, 2017), which is incorporated by reference herein. In more detail, the device 100 uses magnetic induction to transfer power to a power receiver (PRx) in the electronic device to be charged when the device to be charged is placed on the charging device 100 which includes a power transmitter (PTx) (see, e.g., FIG. 6, where the device 100 is positioned on the rear of a smartphone 150). The electronic device to be charged is pictured in FIG. 6 as a smart phone 150, but is not necessarily limited thereto. For example, the electronic device to be charged can be a tablet, miniature tablet or other portable device.

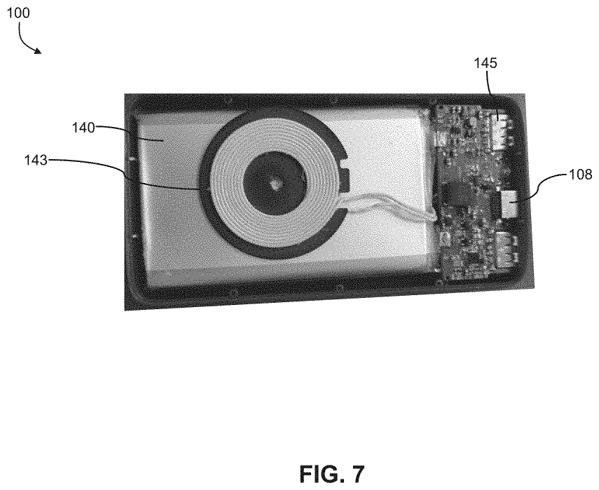

The PTx and PRx subsystems each comprise coils, as well as circuitry that handles the communication and power transfer between the charging device 100 and the device to be charged (see, e.g., FIG. 7 showing an internal component view of the device 100 comprising a battery 140, a PTx coil 143 and a printed circuit board assembly (PCBA) 145). Power is transferred from the PTx in the device 100 to a PRx in the device to be charged. Prior to charging, the PRx and PTx communicate with each other to establish that the device to be charged (e.g., smartphone 150) is capable of being charged by the charging device 100, whether it needs to be charged, how much power is required, etc. The communication ensures an appropriate power transfer from the PTx to the PRx. The magnetic field generated by the charging device 100 is able to be transferred through any non-metallic, non-ferrous materials, such as, for example, plastics, glass, water, wood, and air so that wires and connectors are not needed between the power transmitter and power receiver. In one or more embodiments, the charging device 100 utilizes a 5 V/2-Amp AC adapter in to draw sufficient power from an electrical outlet, as well as a Universal Serial Bus (USB) cable from an adapter to the device 100.

UV-C radiation is a disinfectant for air, water and surfaces that can reduce infection risk. Many bacteria and viruses, including various coronaviruses, respond to UV-C disinfection. In some tests, UV-C light sources inactivated 99% of the SARS-CoV-2 virus on a surface with an exposure time of 6 seconds. UV-C light is in the range of 100 nm-280 nm. According to one or more embodiments, the light sources 103 emit UV-C light with a wavelength of about 270 nm to about 280 nm at a forward voltage in the range of about 5 my to about 7 my and a reverse current of about 5 μA. In one or more embodiments, the light sources 103 further emit UV-A light with a wavelength of about 390 nm to about 400 nm, at a forward voltage in the range of about 3 my to about 3.3 my and a reverse current of about 5 μA. A protective cover 107 is disposed over the light sources 103, and includes, for example, general purpose polystyrene (GPPS) or other similar plastic material.

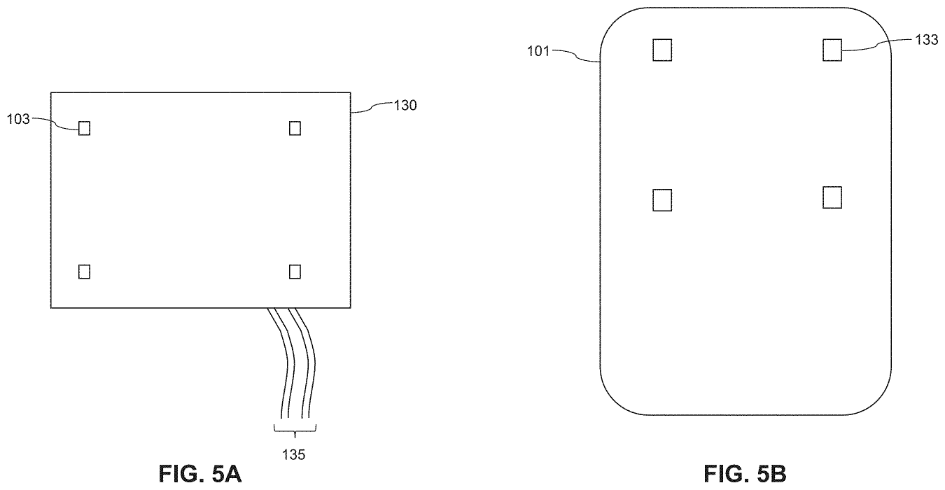

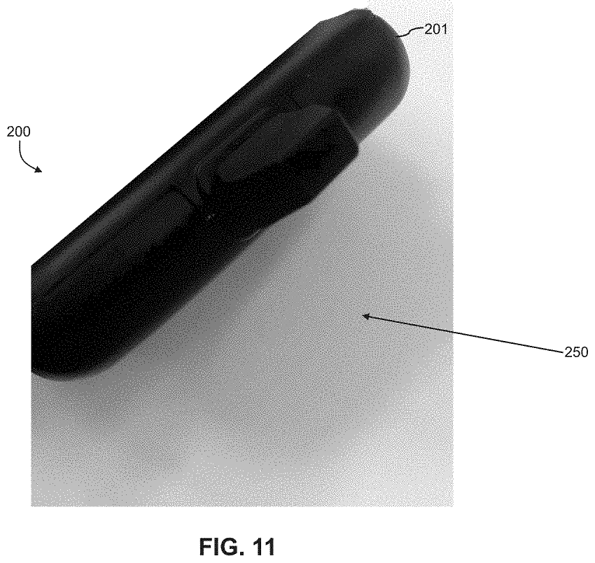

Referring to FIGS. 5A and 5B, in one or more embodiments, the light sources 103 are disposed in the housing 101, and aligned with respective openings 133 in a front face of the housing 101. The light sources 103 are disposed on and coupled to a substrate 130 using, for example, an adhesive. The light sources 103 receive power a battery 140 in the housing 101 and can be turned on an off via wires 135 extending through an opening in the substrate 130. The wires are electrically connected to the battery 140 in the housing 101, and to a switch, which is switched on and off by pressing a button 105 (see FIGS. 1, 3 and 4) for an extended period of time (e.g., “long-press” for 3 seconds). As described further herein in connection with FIG. 11, the light sources 103 emitting UV-C light can be directed to a surface to be sanitized for a recommended period of time (e.g., 6-10 seconds). The device 100 includes a UV driver (no shown) to control operation of the light sources 103.

A rechargeable battery 140 is disposed in the housing 101, and is electrically connected to the PTx and to the light sources 103. The housing 101 comprises a female connector 108 electrically connected to the battery 140, for example, the PCBA 145 and one or more wires. The female connector 108 is configured for receiving, for example, a male micro-USB connector, a male USB-mini connector, a male USB-C connector, a male lightning connector, or other type of connector that is connected to a power source, such as, for example, a standard household outlet providing 15 amperes (amps) of current and 120 volts. For example, the female connector 108 is connected to an outlet via a power cord having a male connector inserted into the female connector 108 at one end and connected at the other end to a plug inserted into the outlet. The power cord is connected to the plug via, for example, a USB connector that is inserted into the plug, which is plugged into the outlet. The battery 140 of the device 100 is able to be charged via the female connector 108 when connected to the power source.

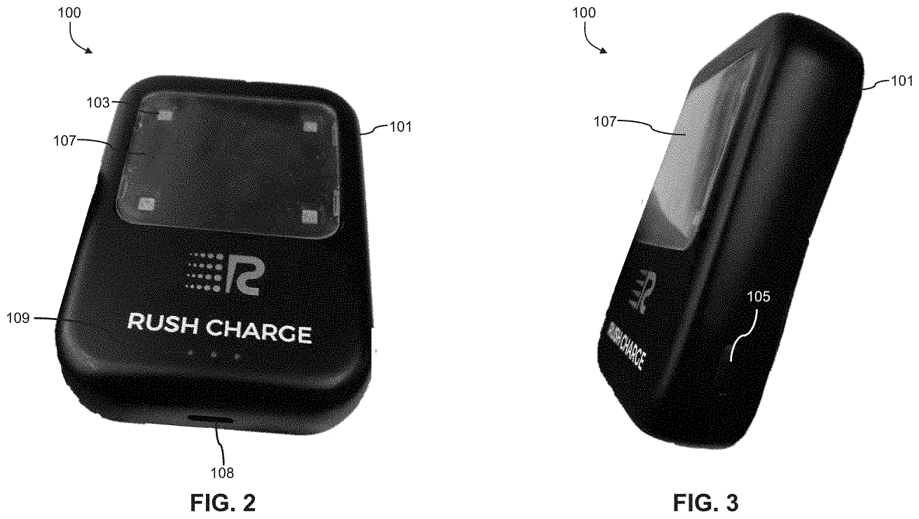

Referring to FIG. 2, the device 100 further includes a plurality of light emitting diodes (LEDs) 109 on the front surface of the housing 101. The LEDs 109 are electrically connected to the PCBA 145 and are connected to circuitry in the PCBA to enable the LEDs 109 to indicate a charge status of the battery 140 in the housing 101. For example, the charge status of the battery 140 can be indicated by a number of the LEDs 109 that are lit at a given time and/or by the color of the LEDs 109. The number of LEDs 109 that are lit is proportional to the amount of charge of the battery (e.g., less lit LEDs 109=less charge, and more lit LEDs 109=more charge). In addition or as an alternative to indicating charge status with the number of lit LEDs 109, a color of the LEDs 109 may indicate an amount of charge for the battery (e.g., green/blue—75-100% charged, yellow—25-50% charged, red—0-25% charged). Other indicators besides LEDs may be used.

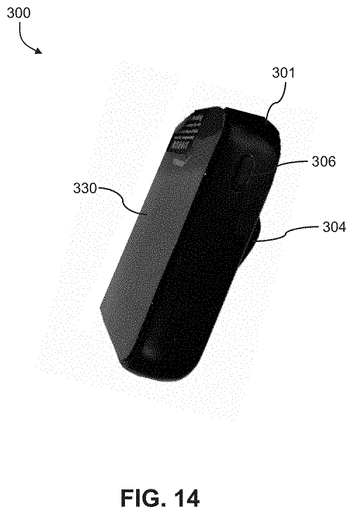

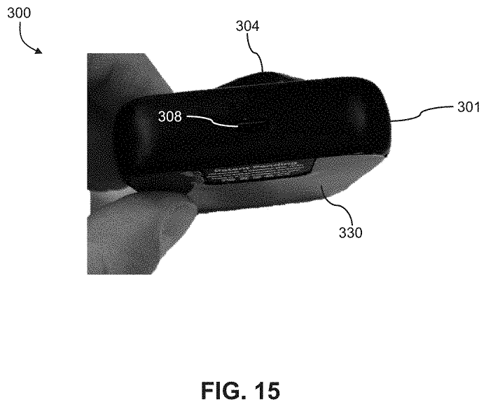

In operation, when the electronic device to be charged (e.g., smartphone 150, tablet or other rechargeable portable personal electronic device) is attached to the device 100, wireless charging occurs as described herein above. In this case, the device 100 via the PTx and a PRx in the device to be charged, is configured for having the battery 140 charge the connected electronic device to be charged. Although not shown, the device 100 includes a reusable double-sided adhesive attached to the rear surface of the device 100 (see, e.g., element 330 of the device 300 described below in connection with FIGS. 14 and 15). The double-sided adhesive keeps items in place without slipping (e.g., the charging device 100 on the smartphone 150) and allows for easy separation of the devices from each other without surface damage. According to an embodiment, the double-sided adhesive comprises Nano (nanotechnology) PU gel or the like.

A single press of the button 105 (e.g., “short-press”) turns on the power to commence charging or will turn on the device 100 from sleep mode. Alternatively, the device 100 will activate automatically when the device 100 is affixed to a device to be charged.

As described above, a power source can be connected to the device 100 via the female connector 108. The power source (e.g., from a wall outlet or other charging device) can be connected to the device 100 while the second electronic device is attached to the device 100 and charging. The device is configured via the PCBA 145 to have pass through technology, such that using the power source, the battery 140 in the device 100 can be recharging while a connected second device is also being charged.

According to an embodiment, the PCBA 145 includes load balancing circuitry and power regulating circuits to transfer power from the power source to the battery 140 in the device 100 and to the connected second device. As can be understood, the device 100 is a portable charging device that can be used to charge other devices while not connected to (e.g., plugged into) a power source, or while connected to (plugged into) a power source.

In accordance with one or more embodiments, the battery 140 in the device 100 is configured to be recharged with suitable power requirements, such as, by non-limiting example, recharging with at least 5V and 1.3 A, which would recharge the battery 140 in approximately 75 minutes. Such a capability allows the portable recharging device 100 to be reused multiple times, such as, for example, over 1,000 times.

According to one or more embodiments, the battery 140 in the device 100 has a capacity to fully recharge the battery of a connected electronic device. By non-limiting example, the battery of the portable recharging device 100 can be 1800 mAh-3600 mAh. The battery 140 is configured to recharge an electronic device with suitable power requirements, such as, by non-limiting example, with the portable recharging device 100 outputting at least 5V and 1.7 A.

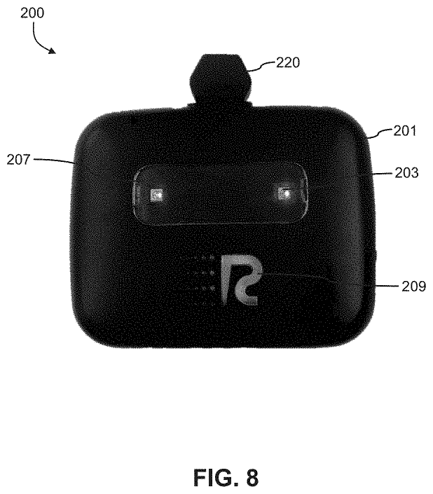

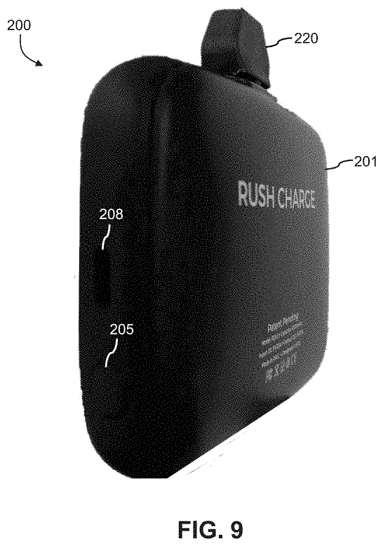

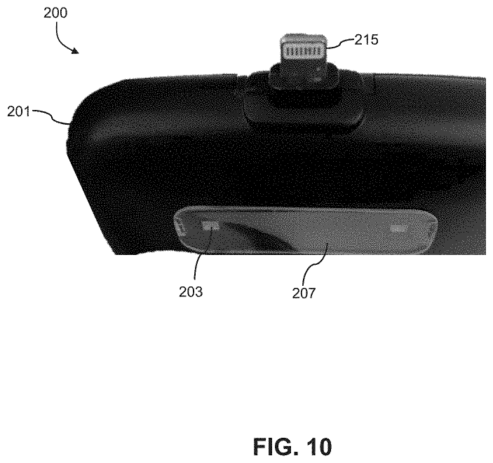

Referring to FIGS. 8-11, another embodiment of a charging and sanitizing device 200 is shown. Similar to the device 100, the device 200 includes a housing 201 and light sources 203. The light sources 203 are configured in the same or similar manner as the light sources 103, and repetitive descriptions of the same or similar components is omitted. However, as can be seen in FIGS. 8 and 10, there are two light sources 203 instead of 4 light sources. The number of light sources is not limited to 4 or 2, and there may be more or less light sources 103 and 203 for each device 100 and 200. The button 205 and the female connector 208 operate in the same or similar manner as the button 105 and female connector 208 described in connection with the device 100.

Referring to FIG. 11, upon activation via the button 205, the light sources 203 emit UV-C light, which can be directed to a surface 250 to be sanitized for a recommended period of time (e.g., 6-10 seconds).

Unlike the device 100, the device 200 does not charge a second electronic device wirelessly, and instead connects to a second electronic device via a male connector 215 that is protected by a cap 220 when not in use. The cap 220 can be made of, for example, rubber or plastic. The male connector 215 extends from the top of the housing 201 and is integrated with the housing 201. The male connector 105 comprises, for example, a male micro-universal serial bus (USB) connector, a male USB-mini connector, a male USB-C connector, a male lightning (Apple®) connector or other type of portable device connector. A rechargeable battery (no shown) is disposed in the housing 201, and is electrically connected to the male connector 215. The male connector 215 is configured for being inserted into a female connector of a second electronic device, including but not necessarily limited to, a smartphone, tablet or other rechargeable portable personal electronic device. When connected to the device 200 via the male connector 215, the battery can charge a battery of the second electronic device.

Similar to the device 100, a power source can be connected to the device 200 via the female connector 208. The power source (e.g., from a wall outlet or other charging device) can be connected to the device 200 while the second electronic device is attached to the device 200 and charging. The device is configured via a PCBA to have pass through technology, such that using the power source, the battery in the device 200 can be recharging while a connected second device is also being charged. In the device 200, an LED configuration 209 lights up a logo (see, e.g., the “R” logo in FIG. 8. The LED configuration 209 includes LEDs which operate in the same or similar manner as the LEDs 109 of FIG. 1, except that the logo is lit up by LEDs in the device 200 to indicate a level of charge of a battery of the device 200.

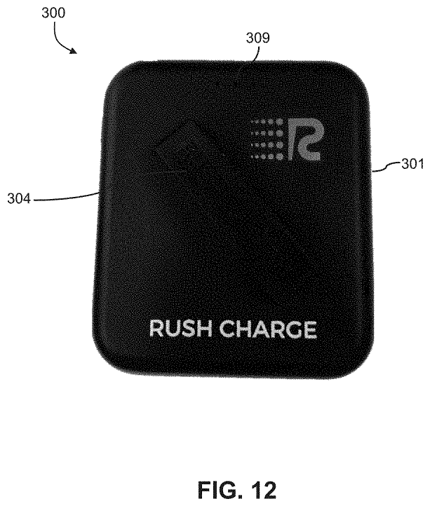

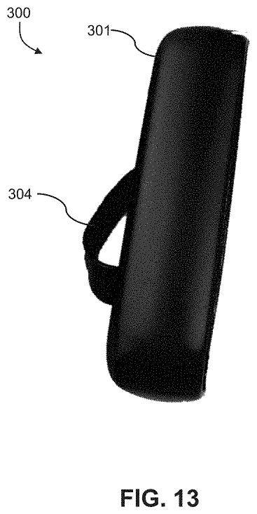

Referring to FIGS. 12-15, an embodiment of a charging device 300 is shown. Similar to the device 100, the device 300 includes a housing 301. The charging device 300 is nota sanitizing device, and does not include sanitizing light sources. However, similar to the device 100, the device 300 is a wireless charging device comprising a PTx and is configured to be attached to a smartphone, tablet or other rechargeable portable personal electronic device to be close enough to the rechargeable portable personal electronic device to allow for wireless charging in accordance with the WPC. Referring to FIGS. 14 and 15, the device 300 includes a re-usable double-sided adhesive 330 attached to the rear surface of the device 300. The double-sided adhesive 330 keeps the device 300 in place on a rear surface of a smartphone, tablet or other rechargeable portable personal electronic device (e.g., smartphone 150) without slipping and allows for easy separation of the devices from each other without surface damage. According to an embodiment, the double-sided adhesive 330 comprises Nano PU gel or the like.

Unlike the device 100, the device 300 comprises a strap 304 on the front surface of the housing 301, which permits a user to insert one or more fingers through the strap 304 so that a user can hold the device 300. For example, a user can hold the device 300 via the strap 304 when the device 300 is attached to a device that is being charged, which enables the user to view the screen of the device being charged in an unobstructed manner while that device is charging. The strap 304 is attached to the front surface of the housing via, for example, an adhesive and/or by ultrasound techniques. A single press of the button 306 turns on the power to commence charging or will turn on the device 300 from sleep mode. Alternatively, the device 300 will activate automatically when the device 300 is affixed to a device to be charged.

Similar to the device 100, a power source can be connected to the device 300 via the female connector 308. The power source (e.g., from a wall outlet or other charging device) can be connected to the device 300 when the device 300 is not charging another device or while the device 300 is charging a second electronic device. The device is configured via a PCBA to have pass through technology, such that using the power source, the battery in the device 300 can be recharging while a second device is also being charged. The LEDs 309 of the device 300 are the same or similar in configuration and operation as the LEDs 109 of the device 100.

In an embodiment, the devices 100, 200 and 300 maintain portability features such that, in a non-limiting example, the devices 100, 200 and 300 are small enough to fit in one's pocket for storage.

Although one male connector 215 is shown in device 200 and one female connector 108, 208 and 308 are shown in the devices 100, 200 and 300, the embodiments are not necessarily limited thereto. The devices 100, 200 and 300 may include more than one male and/or female connectors for different types of connections. For example, the devices 100, 200 and 300 can include connectors for both Android® and Apple® portable devices.

Although exemplary embodiments of the present invention have been described hereinabove, it should be understood that the present invention is not limited to these embodiments, but may be modified by those skilled in the art without departing from the spirit and scope of the present invention.

What is claimed is:

1. An electronic device, comprising:
a housing;
a battery in the housing;
a power transmitter in the housing configured for wireless charging of a second electronic device; and
at least one light source emitting ultraviolet-C (UV-C) light.

2. The electronic device according to claim 1, further comprising a reusable adhesive portion on a rear surface of the housing.

3. The electronic device according to claim 2, wherein the electronic device is configured to be attached to the second electronic device via the reusable adhesive portion.

4. The electronic device according to claim 1, wherein the UV-C light comprises a wavelength in a range of 270 nm to about 280 nm.

5. The electronic device according to claim 1, further comprising a female connector electrically connected to the battery.

6. The electronic device according to claim 5, wherein the battery is configured to be charged via a source of electrical power connected through the female connector.

7. The electronic device according to claim 5, wherein the female connector is disposed in the housing.

8. The electronic device according to claim 1, wherein the electronic device is portable.

9. An electronic device, comprising:
a connector for connecting to a second electronic device;
a battery configured for charging another battery of the connected second electronic device; and
at least one light source emitting ultraviolet-C (UV-C) light.

10. The electronic device according to claim 9, wherein the UV-C light comprises a wavelength in a range of 270 nm to about 280 nm.

11. The electronic device according to claim 9, further comprising a female connector electrically connected to the battery.

12. The electronic device according to claim 11, wherein the battery is configured to be charged via a source of electrical power connected through the female connector.

13. The electronic device according to claim 11, wherein the female connector is disposed a housing of the electronic device.

14. The electronic device according to claim 9, wherein the electronic device is portable.

15. The electronic device according to claim 9, wherein the connector comprises one of a male micro-USB connector, a male USB-mini connector, a male USB-C connector, and a male lightning connector.
